(19) United States
(12) Patent Application Publication
(10) Pub. No.: US 2023/0182042 A1
(43) Pub. Date: Jun. 15, 2023

(54) DEVICE AND METHOD FOR SEPARATING GAS BUBBLES FROM A LIQUID FLOW

(71) Applicant: MEDTRON AG, D-66128 Saarbrucken (DE)

(72) Inventors: Stefan Kreber, Saarbrucken (DE); Michael SCHWEIGMANN, Zweibrucken (DE); Hanno ALTMEYER, Neunkirchen (DE)

(21) Appl. No.: 17/993,198

(22) Filed: Nov. 23, 2022

(30) Foreign Application Priority Data
Nov. 25, 2021 (EP) 212104301

(51) Int. Cl. B01D 19/00

(52) U.S. Cl. CPC B01D 19/0042

(57) ABSTRACT

A device for separating gas bubbles from a liquid flow, having a degassing chamber with an inlet conduit and an outlet conduit for conducting the liquid flow and having a collecting chamber that communicates with the degassing chamber and having a gas outlet opening for the gas bubbles, wherein the degassing chamber includes a ring conduit that extends in an arc shape in a vertical plane and connects the inlet conduit to the outlet conduit in a flow direction and the collecting chamber is positioned in a vertically upper region of the ring conduit. A corresponding method for separating gas bubbles from a liquid flow is also disclosed.

### BACKGROUND OF THE INVENTION

### Field of the Invention

This invention relates to a device for separating gas bubbles from a liquid flow, including a degassing chamber having an inlet conduit and an outlet conduit for conducting the liquid flow and having a collecting chamber that communicates with the degassing chamber and has a gas outlet opening for the gas bubbles. This invention also relates to a method for separating gas bubbles from a liquid flow.

### DISCUSSION OF RELATED ART

Known devices and methods are used to separate undesirable gas components in the form of gas bubbles entrained in a liquid flow and to discharge them separately from the liquid flow. For example, in medical applications in which a liquid supplied from an injector is injected into a patient’s blood vessel, such a separation of gas bubbles is particularly important because injecting gas bubbles into the bloodstream can potentially result in life threatening complications for the patient. Examples of such liquid flows administered with injectors include contrast medium and/or saline solution injections as part of a medical imaging exam, for example, computed tomography (CT) or magnetic resonance tomography (MRT).

In such applications, there is a desire for a device and a method that reliably ensure the separation of gas bubbles from the liquid flow, can be easily used by medical personnel, and furthermore, particularly when used as a single-use article, can be produced for a low manufacturing cost, such as a magnetic resonance imaging system, and it is also necessary to avoid using metallic materials because of the powerful magnetic fields involved.

One object of this invention is to provide a device and method of the type mentioned above, which are particularly suitable for the explained application purposes in the medical field.

In order to attain this object and others, this invention provides a device according to the features described in this specification and in the claims.

### SUMMARY OF THE INVENTION

Advantageous embodiments and modifications of this invention are also described in this specification and in the claims.

One embodiment of this invention provides a device in which the degassing chamber has a ring conduit extending in an arc, preferably a circular arc, in a vertical plane and connecting the inlet conduit to the outlet conduit in a flow direction and the collecting chamber is positioned in the vertically upper region, for example, adjacent to the highest point or at the highest point of the ring conduit.

In the context of this invention, with such a ring conduit positioned in a vertical plane and connecting the inlet conduit to the outlet conduit in an arc shape, a particularly effective separation of entrained gas bubbles from the liquid flow can be achieved.

This can be at least partially attributed to the ring conduit following the inlet conduit in the flow direction, the liquid flow, which is supplied by the latter and contains gas bubbles, and is deflected in an arc-shaped fashion with a specific minimum flow velocity in accordance with the curvature thereof, wherein a velocity gradient of the liquid flow and the entrained gas bubbles is established across a width of the flow cross-section through the ring conduit. In radially outer regions of the ring conduit extending in a circular arc shape, for example, adjoining or adjacent to the outer diameter of the ring conduit, higher flow velocities of the liquid flow including the entrained gas bubbles occur than in the radially inner regions of the ring conduit extending in an arc shape, for example, adjoining the inner diameter of the ring conduit. In this connection, it should be noted that entrained large-volume gas bubbles, because the ring conduit extends in an arc shape in a vertical plane and because of the lower density of these bubbles relative to the liquid flow, quickly rise vertically to the outer diameter of the ring conduit and from there, pass over into the collecting chamber positioned in the vertically upper region of the ring conduit. From there, the gas bubbles exiting the liquid flow are discharged separately from the liquid flow via a corresponding gas outlet opening.

The liquid flow that is thus freed of the large-volume gas bubbles is guided in the ring conduit in the direction of the outlet conduit.

At the same time, however, due to the prevailing velocity gradient of the liquid flow, the small-volume gas bubbles that are still present, which do not rise at a high velocity in the liquid flow because of their small volume and are not supplied directly to the collecting chamber, are deflected radially inward inside ring conduit extending in an arc shape by the prevailing combination of centrifugal force and density difference as a result of which their flow velocity in the flow direction decreases further. As soon as the small-volume gas bubbles come into contact with the top surface that constitutes or forms the inner diameter of the ring conduit extending in an arc shape, they collect at this top surface and gradually agglomerate to form large-volume gas bubbles that are made up of a large number of small-volume gas bubbles and that in the end, have a volume that is large enough that they are able to automatically rise to the top vertically and from there, pass over into the collecting chamber.

Tests have shown that a variety of volume sizes of gas bubbles in a liquid flow can be reliably removed in this way, and the effectiveness is even greater with the higher the flow velocity of the liquid flow through the ring conduit. This fits in very well with medical applications such as those using injection since, for example, in angiographic tests with injection pressures of up to 83 bar and correspondingly high flow velocities of the liquid flow, rates of up to about 40 ml/s are used.

The differentiation into large-volume and small-volume gas bubbles is dependent on the respective density ratios. For purposes of this invention, large-volume gas bubbles are understood to mean those gas bubbles that automatically rise in the liquid flow and pass over into the collecting chamber. If the buoyancy force of smaller gas bubbles is insufficient for such an automatic rising into the collecting chamber, then for purposes of this invention, they are considered to be small-volume gas bubbles. The lower limit for small-volume gas bubbles is a size that can be injected into the bloodstream of a patient with no danger.

To further increase the flow velocity, according to one embodiment of this invention, the cross-section of the ring conduit tapers in the flow direction so that the flow velocity increases from the inlet conduit to the outlet conduit.

According to another embodiment of this invention, the ring conduit extends continuously over 360° in the vertical plane and the inlet conduit feeds into the ring conduit tangentially and before the collecting chamber in the flow direction while the outlet conduit leads radially outward from the ring conduit after the collecting chamber and before the mouth of the inlet conduit, when viewed in the flow direction. Such an arrangement makes it possible for only a partial quantity of the liquid flow that is guided in the ring conduit, after having been supplied via the inlet conduit, to be immediately discharged again via the outlet conduit. The remaining amount of the liquid guided in the ring conduit, however, is guided for a full 360° revolution in the ring conduit and passes through the latter at least two times until it is finally discharged by the outlet conduit. Such an arrangement makes it possible according to this invention to increase the efficiency of the separation of entrained gas bubbles even further.

In order to ensure a homogeneous mixing of the partial quantity of the liquid flow that has been guided for a full revolution in the ring conduit together with the liquid flow that has just been supplied into the ring conduit by the inlet conduit, it is also possible, preferably directly before the mouth of the inlet conduit, when viewed in the flow direction, for an overflow stage to be provided, which adjoins the mouth of the inlet conduit and merges the liquid flow that has been guided for the full revolution together with and layered over or under the liquid flow supplied by the inlet conduit.

According to another embodiment of this invention, the ring conduit is delimited by an essentially arc-shaped inner wall and an arc-shaped outer wall positioned concentric thereto, with the latter being embodied as interrupted in the region of the mouths of the inlet conduit and outlet conduit and of the collecting chamber. The arc-shaped inner wall by contrast is preferably embodied as continuous.

According to another embodiment of this invention, the above-explained degassing chamber is positioned in an insert housing, which can be used, for example, as a replaceable single-use part in a corresponding receiving housing of a medical injector and in the region of or near the inlet conduit, the outlet conduit, and the gas outlet opening, is connected to corresponding connections of the medical injector when it is inserted into the receiving housing.

One method for separating gas bubbles from a liquid flow that is proposed in connection with this invention is based on the fact that the liquid flow is guided in a ring conduit starting from an inlet conduit to an outlet conduit in a flow direction extending in an arc shape, preferably an essentially circular arc shape, in a vertical plane. According to this invention, as a result of the liquid flow being guided in the ring conduit together with the entrained gas bubbles in an arc shape in the vertical plane, large-volume gas bubbles rise in the vertical direction to a degassing chamber positioned in the vertically upper region, for example, adjoining or adjacent to the highest point or at the highest point of the ring conduit and are discharged from there while the remaining small-volume gas bubbles are deflected radially inward and collect in the region of or near an inner wall of the ring conduit and agglomerate to form large-volume gas bubbles and then rise to the degassing chamber and are discharged.

According to this invention, during this, the liquid flow can be accelerated in the flow direction, for example, through a gradual change in the available flow cross-section in the ring conduit.

Also, it is possible for only a partial quantity of the liquid flow that is guided in the ring conduit to be immediately discharged from the ring conduit by the outlet conduit, and the remaining partial quantity remains in the ring conduit for a full revolution and flows through it again.

### BRIEF DESCRIPTION OF THE DRAWINGS

Other embodiments and details of the device according to this invention and of the method according to this invention are explained below in conjunction with an exemplary embodiment shown in the drawings, wherein:

FIG. 1 shows a top view of a device according to one embodiment of this invention; and

FIG. 2 shows a perspective view of the device according to FIG. 1.

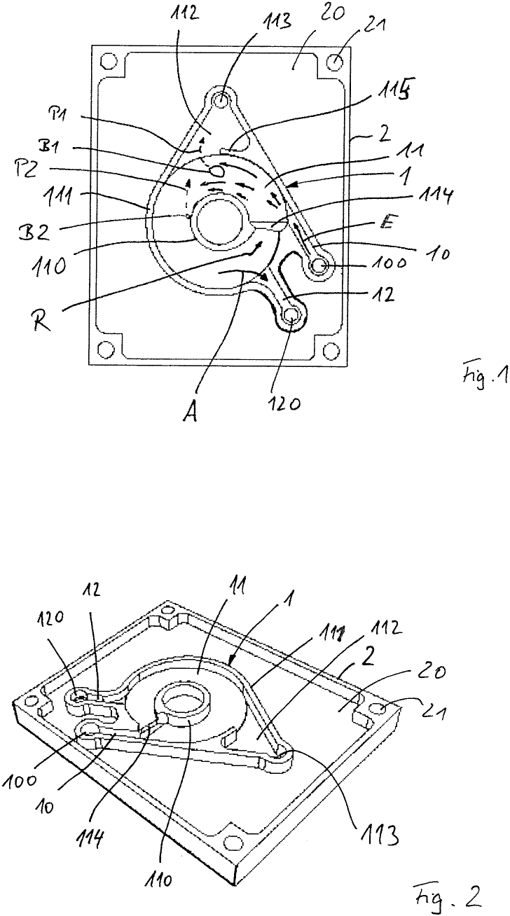

### DETAILED DESCRIPTION OF THE INVENTION

The Figures show a device for separating gas bubbles from a liquid flow, which can be produced, for example, as a one-piece single-use part made of an injection-molded, printed, or milled plastic and is particularly suitable for medical applications under corresponding hygienic conditions.

One essential component of the device shown in the Figures is a degassing chamber 1, which will be explained in greater detail below and is enclosed by an insert housing 2 that is embodied here as block-shaped and comprises a back wall 20, which extends in a vertical plane in the installed position shown, and a front wall, which is removed here and is affixed in bores 21, between which the degassing chamber 1 is positioned. The device is used in this vertical orientation shown.

The degassing chamber 1 comprises an inlet opening 100 that passes through the back wall 20 for a liquid flow such as, in the case of a medical injector, a contrast medium and/or saline solution that is injected from it in the direction toward a patient and that enters the degassing chamber 1 via an inlet conduit 10 according to arrow E. Adjacent to the inlet conduit 10, the degassing chamber 1 comprises a ring conduit 11 that communicates with the inlet conduit 10 and in the exemplary embodiment shown extends first upward and then downward in a circular arc in the vertical plane. The mouth of the inlet conduit 10 into the ring conduit 11, which is embodied with a much larger flow cross-section than it, occurs tangentially, approximately at the 3:00 o′clock position, and is directed vertically upward.

The ring conduit 11 is delimited by an uninterrupted cylindrical inner wall 110 and an outer wall 111 positioned concentric thereto, with the latter being embodied as interrupted on the one hand by the mouth of the inlet conduit 10 and on the other hand by the respective mouths, which will be explained in greater detail below, of an outlet conduit 12 and in the vertically upper region in which the initially upward-directed path of the ring conduit transitions into a downward-directed path of a collecting chamber 112, which is thus positioned between the mouths of the inlet conduit 10 and outlet conduit 12.

The collecting chamber 112 is embodied as approximately cupola-shaped in the vertically upper region of the ring conduit 11, is positioned adjoining the latter’s outer circumference and highest point, and is separated from the ring conduit 11 by a partition wall 115 provided with an opening. In the vertically lower region approximately at the 5:00 o′clock position, the outlet conduit 12 extends radially outward from the ring conduit 11. The outlet conduit 12 communicates with a connecting line, not shown here, to the patient via an outlet bore 120 leading out of the insert piece 2 and its back wall 20.

The collecting chamber 112 positioned in the vertically upper part is likewise provided with a gas outlet opening 113 that passes through the back wall 20.

When the degassing chamber 1 is charged with the liquid flow supplied via the inlet conduit 10, it travels into the ring conduit 11 and as a result, is forced into a circular path extending in a vertical plane following the arc-shaped path of the ring conduit 11 in the vertical plane. Entrained large-volume gas bubbles B1, which have a volume of such a kind that they can automatically rise due to a density difference in the liquid flow, travel directly into the collecting chamber 112 positioned vertically above the ring conduit 11 according to arrow P1 and are discharged via the gas outlet opening 113. The liquid flow, however, is guided further on the circular path along the partition wall 115 and in a flow direction along the ring conduit 11 in the direction of the outlet conduit 12.

Entrained small-volume gas bubbles B2 whose buoyancy is insufficient for them to rise directly into the collecting chamber 112 are pushed against and end up adhering to the inner wall 110 of the ring conduit 11 by a velocity gradient of the flow velocity of the liquid flow that is established in the ring conduit 11. The velocity gradient is demonstrated by different arrow lengths in the ring conduit 11, and higher flow velocities prevail in the radially outer region of the ring conduit 11 than in the radially inner region of the ring conduit and almost come to a standstill in the region of the inner wall 110. As a result, the small-volume gas bubbles B2 gradually collect against the inner wall 110 and merge with one another, for example, they agglomerate and they increase in volume until they assume an overall volume of such a kind that they can automatically rise into the collecting chamber 112 according to arrow P1.

The liquid flow that is freed of gas bubbles as it circulates through the ring chamber 11, after approximately ¾ of a revolution, is then at least partially discharged according to arrow A by the radially outward-leading outlet conduit 12 and the adjoining outlet bore 120, whereas the remaining part of the liquid flow executes a full revolution in the ring conduit 11 according to arrow R and once again joins a liquid flow that is supplied by the inlet conduit 10 in order to then complete a new revolution together with it in the ring conduit 11 with a separation of additional gas bubbles.

In order to prevent the occurrence of turbulence in the region of or near the merging of the partial flow that has completed a full revolution according to arrow R and the additional liquid flow supplied via the inlet conduit 10 according to arrow E, an overflow stage 114 is provided in the ring conduit 11 just before the mouth of the inlet conduit 10 into the ring conduit 11 viewed in the flow direction, which ensures that two layers form in the ring conduit 11, namely one layer including the liquid flow coming in via the inlet conduit 10 according to arrow E and on top of this, another layer including the partial flow according to arrow R, which has completed a full revolution in the ring conduit 11.

It has turned out that a simple device of this kind, which performs without moving parts, can be used to reliably separate a liquid flow from entrained gas bubbles, particularly at high flow velocities, wherein the energy required for this can be obtained solely from the flow velocity of the liquid flow.

While in the foregoing specification this invention has been described in relation to certain preferred embodiments, and many details are set forth for purpose of illustration, it will be apparent to those skilled in the art that this invention is susceptible to additional embodiments and that certain of the details described in this specification and in the claims can be varied considerably without departing from the basic principles of this invention.

1-12. (canceled)

13. A device for separating gas bubbles from a liquid flow, comprising a degassing chamber having an inlet conduit and an outlet conduit for conducting the liquid flow and having a collecting chamber communicating with the degassing chamber and having a gas outlet opening for the gas bubbles, the degassing chamber comprising a ring conduit extending in an arc shape in a vertical plane and connecting the inlet conduit to the outlet conduit in a flow direction and the collecting chamber positioned in a vertically upper region of the ring conduit.

14. The device according to claim 13, wherein the ring conduit extends in a circular arc shape in the vertical plane.

15. The device according to claim 14, wherein a cross-section of the ring conduit tapers in the flow direction.

16. The device according to claim 15, wherein the ring conduit extends continuously over 360°, the inlet conduit feeds tangentially into the ring conduit, and the outlet conduit leads radially outward from the ring conduit before the mouth of the inlet conduit when viewed in the flow direction.

17. The device according to claim 16, wherein the ring conduit has an overflow stage that adjoins the mouth of the inlet conduit before the mouth of the inlet conduit, when viewed in the flow direction.

18. The device according to claim 17, wherein the ring conduit is delimited by an essentially arc-shaped inner wall and an outer wall positioned concentric thereto and being embodied as interrupted in the region of the mouths of the inlet conduit and outlet conduit and of the collecting chamber.

19. The device according to claim 18, wherein the degassing chamber is positioned in an insert housing.

20. A medical injector for injecting a liquid flow into a patient, comprising a device according to claim 1.

21. A method for separating gas bubbles from a liquid flow in which the liquid flow containing the gas bubbles is guided in a ring conduit starting from an inlet conduit to an outlet conduit in a flow direction extending in an arc shape in a vertical plane, wherein large-volume gas bubbles rise in a vertical direction to a degassing chamber and are discharged and the remaining small-volume gas bubbles are deflected radially inward and collect in the region of an inner wall of the ring conduit and agglomerate to form large-volume gas bubbles and rise to the degassing chamber and are discharged.

22. The method according to claim 21, wherein the liquid flow containing the gas bubbles in the annular conduit is guided in an essentially circular arc-shaped fashion in the vertical plane.

23. The method according to claim 22, wherein the liquid flow is accelerated in the flow direction.

24. The method according to claim 23, wherein only a partial quantity of the liquid flow that is guided in the ring conduit is discharged from the ring conduit by the outlet conduit and the remaining partial quantity remains in the ring conduit for a full revolution and flows through it again.

25. The device according to claim 13, wherein a cross-section of the ring conduit tapers in the flow direction.

26. The device according to claim 13, wherein the ring conduit extends continuously over 360°, the inlet conduit feeds tangentially into the ring conduit, and the outlet conduit leads radially outward from the ring conduit before the mouth of the inlet conduit when viewed in the flow direction.

27. The device according to claim 26, wherein the ring conduit has an overflow stage that adjoins the mouth of the inlet conduit before the mouth of the inlet conduit, when viewed in the flow direction.

28. The device according to claim 13, wherein the ring conduit is delimited by an essentially arc-shaped inner wall and an outer wall positioned concentric thereto and being embodied as interrupted in the region of the mouths of the inlet conduit and outlet conduit and of the collecting chamber.

29. The method according to claim 21, wherein the liquid flow is accelerated in the flow direction.

30. The method according to claim 21, wherein only a partial quantity of the liquid flow that is guided in the ring conduit is discharged from the ring conduit by the outlet conduit and the remaining partial quantity remains in the ring conduit for a full revolution and flows through it again.
